(19) United States
(12) Patent Application Publication
(10) Pub. No.: US 2006/0093728 A1
(43) Pub. Date: May 4, 2006

(54) Process for hydrating lenses

(75) Inventors: Sandeep Pandit, Rochester, NY (US); Sanjay Rastogi, Rochester, NY (US)

Correspondence Address: Bausch & Lomb Incorporated, One Bausch & Lomb Place, Rochester, NY, 14604-2701, US

(73) Assignee: Bausch & Lomb Incorporated

(21) Appl. No.: 11/230,132

(22) Filed: Sep. 19, 2005

Related U.S. Application Data
(60) Provisional application No. 60/624,119, filed on Nov. 1, 2004.

(51) Int. Cl. A61L 33/00; B05D 5/06

(52) U.S. Cl. 427/2.1; 427/162

(57) ABSTRACT

A process for hydrating biomedical devices, particularly ophthalmic devices including contact lenses, intraocular lenses and ophthalmic implants, involves exposing the devices to condensed water vapor.

This application claims the benefit under 35 USC 119(e) of Provisional Patent Application No. 60/624,119, filed Nov. 1, 2004.

### FIELD OF THE INVENTION

The present invention relates to a process for hydrating biomedical devices, particularly ophthalmic devices including contact lenses, intraocular lenses and ophthalmic implants.

### BACKGROUND OF THE INVENTION

Hydrogels represent a desirable class of materials for the manufacture of various biomedical devices, including contact lenses and intraocular lenses. A hydrogel is a hydrated cross-linked polymeric system that contains water in an equilibrium state. Hydrogel lenses offer desirable biocompatibility and comfort.

In a typical process for the manufacture of hydrogel lenses, a composition containing a mixture of lens-forming monomers is polymerized to obtain a lens. At this stage, the polymeric lens is in the form of a xerogel, i.e., an unhydrated hydrogel. The polymeric xerogel lens is typically hydrated by immersing the lens in an aqueous hydrating composition, composed of water or an aqueous solution, such as buffered saline solution or an aqueous solution containing a surfactant. Generally, in commercial manufacturing processes, a lens is immersed several times in a hydrating composition, i.e., the hydration process involves several cycles of exposing the lens to a hydrating composition. Sometimes, the hydration process involves cycles employing different hydrating compositions. The hydration process serves not only to hydrate the polymeric xerogel lens, but also to rinse debris from the lens and remove undesired water-soluble contaminants from the polymeric material; this is especially true for the initial cycles of the hydration process. After the lens is hydrated, it is typically packaged in a saline solution.

### SUMMARY OF THE INVENTION

This invention provides an improved process for hydrating biomedical devices, particularly ophthalmic devices including contact lenses, intraocular lenses and ophthalmic implants. The process involves exposing the devices to condensed water vapor. According to preferred embodiments, the process comprises: heating a water source in a lower section of a vessel to generate water vapor; suspending biomedical devices in the vessel above the water source; and condensing the water vapor in an upper section of the vessel such that the devices are hydrated with the condensed water vapor. The water vapor condenses on the devices and/or the water vapor condenses above the devices and drips on the devices. The condensed water is absorbed by the polymeric material forming the devices, such that the devices are hydrated. Excess condensed vapor washes not absorbed by the polymeric material washes contaminants from the device.

### BRIEF DESCRIPTION OF THE DRAWING

FIG. 1 is a schematic representation of an apparatus according to various preferred embodiments of this invention.

### DETAILED DESCRIPTION OF VARIOUS PREFERRED EMBODIMENTS

The present invention provides a method for hydrating biomedical devices, especially ophthalmic biomedical devices. The term “biomedical device” means a device intended for direct contact with living tissue. The term “ophthalmic biomedical device” means a device intended for direct contact with ophthalmic tissue, including contact lenses, intraocular lenses and ophthalmic implants. In the following description, the process is discussed with particular reference to hydrogel contact lenses, a preferred embodiment of this invention, but the invention may be employed for extraction of other polymeric biomedical devices.

A hydrogel is a hydrated cross-linked polymeric system that contains water in an equilibrium state. Hydrogel lenses are generally formed by polymerizing a mixture of lens-forming monomers including at least one hydrophilic monomer. Hydrophilic lens-forming monomers include: unsaturated carboxylic acids such as methacrylic acid and acrylic acid; (meth)acrylic substituted alcohols or glycols such as 2-hydroxyethyl methacrylate, 2-hydroxyethyl acrylate, and glyceryl methacrylate; vinyl lactams such as N-vinyl-2-pyrrolidone; and acrylamides such as methacrylamide and N,N-dimethylacrylamide. Other hydrophilic monomers are well known in the art.

The monomer mixture generally includes a crosslinking monomer, a crosslinking monomer being defined as a monomer having multiple polymerizable functionalities. One of the hydrophilic monomers may function as a crosslinking monomer or a separate crosslinking monomer may be employed. Representative crosslinking monomers include: divinylbenzene, allyl methacrylate, ethylene glycol dimethacrylate, tetraethyleneglycol dimethacrylate, polyethyleneglycol dimethacrylate, and vinyl carbonate derivatives of the glycol dimethacrylates.

One class of hydrogels is silicone hydrogels, wherein the lens-forming monomer mixture includes, in addition to a hydrophilic monomer, at least one silicone-containing monomer. When the silicone-containing monomer includes multiple polymerizable radicals, it may function as the crosslinking monomer. This invention is particularly suited for extraction of silicone hydrogel biomedical devices. Generally, unreacted silicone-containing monomers, and oligomers formed from these monomers, are hydrophobic and more difficult to extract from the polymeric device. Therefore, efficient extraction generally requires treatment with an organic solvent such as isopropanol.

One suitable class of silicone containing monomers include known bulky, monofunctional polysiloxanylalkyl monomers represented by Formula (I):

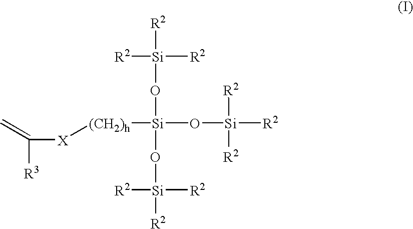

X denotes —COO—, —CONR⁴—, —OCOO—, or —OCONR⁴— where each where R⁴is H or lower alkyl; R³ denotes hydrogen or methyl; h is 1 to 10; and each R² independently denotes a lower alkyl or halogenated alkyl radical, a phenyl radical or a radical of the formula
−Si(R⁵)₃
wherein each R⁵ is independently a lower alkyl radical or a phenyl radical. Such bulky monomers specifically include methacryloxypropyl tris(trimethylsiloxy)silane, pentamethyldisiloxanyl methylmethacrylate, tris(trimethylsiloxy)methacryloxy propylsilane, methyldi(trimethylsiloxy)methacryloxymethyl silane, 3-[tris(trimethylsiloxy)silyl]propyl vinyl carbamate, and 3-[tris(trimethylsiloxy)silyl]propyl vinyl carbonate.

Another suitable class is multifunctional ethylenically “end-capped” siloxane-containing monomers, especially difunctional monomers represented Formula (II): 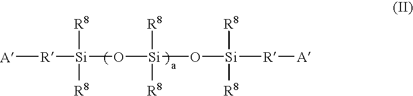
wherein:

each A′ is independently an activated unsaturated group;

each R′ is independently are an alkylene group having 1 to 10 carbon atoms wherein the carbon atoms may include ether, urethane or ureido linkages therebetween;

each R⁸ is independently selected from monovalent hydrocarbon radicals or halogen substituted monovalent hydrocarbon radicals having 1 to 18 carbon atoms which may include ether linkages therebetween, and

a is an integer equal to or greater than 1. Preferably, each R⁸ is independently selected from alkyl groups, phenyl groups and fluoro-substituted alkyl groups. It is further noted that at least one R⁸ may be a fluoro-substituted alkyl group such as that represented by the formula:
-D′-(CF₂)ₛ-M′
wherein:

D′ is an alkylene group having 1 to 10 carbon atoms wherein said carbon atoms may include ether linkages therebetween;

M′ is hydrogen, fluorine, or alkyl group but preferably hydrogen; and

s is an integer from 1 to 20, preferably 1 to 6.

With respect to A′, the term “activated” is used to describe unsaturated groups which include at least one substituent which facilitates free radical polymerization, preferably an ethylenically unsaturated radical. Although a wide variety of such groups may be used, preferably, A′ is an ester or amide of (meth)acrylic acid represented by the general formula: 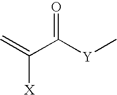
wherein X is preferably hydrogen or methyl, and Y is —O—or —NH—. Examples of other suitable activated unsaturated groups include vinyl carbonates, vinyl carbamates, fumarates, fumaramides, maleates, acrylonitryl, vinyl ether and styryl. Specific examples of monomers of Formula (II) include the following: 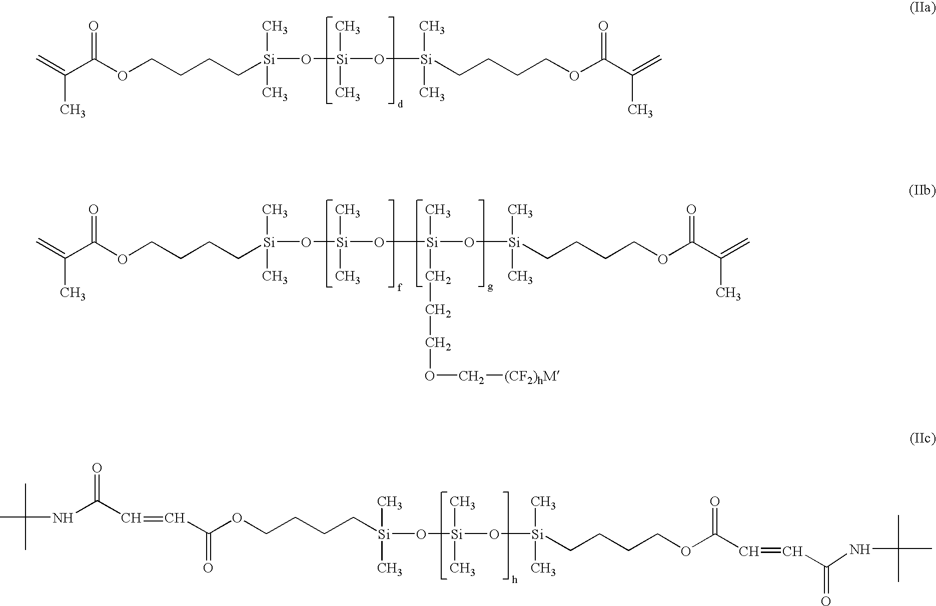
wherein:

d, f, g and k range from 0 to 250, preferably from 2 to 100; h is an integer from 1 to 20, preferably 1 to 6; and

M′ is hydrogen or fluorine.

A further suitable class of silicone-containing monomers includes monomers of the Formulae (IIIa) and (IIIb):
E′(*D*A*D*G)ₐ*D*A*D*E′; or (IIIa)
E′(*D*G*D*A)ₐ*D*G*D*E′; (IIIb)
wherein:

D denotes an alkyl diradical, an alkyl cycloalkyl diradical, a cycloalkyl diradical, an aryl diradical or an alkylaryl diradical having 6 to 30 carbon atoms;

G denotes an alkyl diradical, a cycloalkyl diradical, an alkyl cycloalkyl diradical, an aryl diradical or an alkylaryl diradical having 1 to 40 carbon atoms and which may contain ether, thio or amine linkages in the main chain;

* denotes a urethane or ureido linkage;

a is at least 1;

A denotes a divalent polymeric radical of the formula: 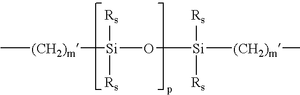
wherein:

each R^{z} independently denotes an alkyl or fluoro-substituted alkyl group having 1 to 10 carbon atoms which may contain ether linkages between carbon atoms;

m′ is at least 1; and

p is a number which provides a moiety weight of 400 to 10,000;

each E′ independently denotes a polymerizable unsaturated organic radical represented by the formula: 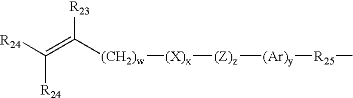
wherein:

R₂₃ is hydrogen or methyl;

R₂₄ is hydrogen, an alkyl radical having 1 to 6 carbon atoms, or a —CO—Y—R₂₆ radical wherein Y is —O—, —S— or —NH—;

R₂₅ is a divalent alkylene radical having 1 to 10 carbon atoms; R₂₆ is a alkyl radical having 1 to 12 carbon atoms; X denotes —CO— or —OCO—; Z denotes —O— or —NH—;

Ar denotes an aromatic radical having 6 to 30 carbon atoms; w is 0 to 6; x is 0 or 1; y is 0 or 1; and z is 0 or 1.

A specific urethane monomer is represented by the following: 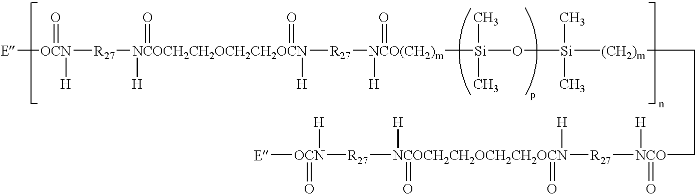
wherein m is at least 1 and is preferably 3 or 4, a is at least 1 and preferably is 1, p is a number which provides a moiety weight of 400 to 10,000 and is preferably at least 30, R₂₇ is a diradical of a diisocyanate after removal of the isocyanate group, such as the diradical of isophorone diisocyanate, and each E″ is a group represented by:

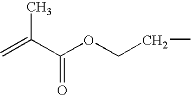

Other silicone-containing monomers include the silicone-containing monomers described in U.S. Pat. Nos. 5,034,461, 5,070,215, 5,260,000, 5,610,252 and 5,496,871, the disclosures of which are incorporated herein by reference. Other silicone-containing monomers are well known in the art.

An organic diluent may be included in the initial monomeric mixture. As used herein, the term “organic diluent” encompasses organic compounds that are substantially unreactive with the components in the initial mixture, and are often used to minimize incompatibility of the monomeric components in this mixture. Representative organic diluents include: monohydric alcohols, such as C₆-C₁₀ monohydric alcohols; diols such as ethylene glycol; polyols such as glycerin; ethers such as diethylene glycol monoethyl ether; ketones such as methyl ethyl ketone; esters such as methyl heptanoate; and hydrocarbons such as toluene.

Generally, the monomer mixtures may be charged to a mold, and then subjected to heat and/or light radiation, such as UV radiation, to effect curing, or free radical polymerization, of the monomer mixture in the mold. Various processes are known for curing a monomeric mixture in the production of contact lenses or other biomedical devices, including spincasting and static casting. Spincasting methods involve charging the monomer mixture to a mold, and spinning the mold in a controlled manner while exposing the monomer mixture to light. Static casting methods involve charging the monomer mixture between two mold sections forming a mold cavity providing a desired article shape, and curing the monomer mixture by exposure to heat and/or light. In the case of contact lenses, one mold section is shaped to form the anterior lens surface and the other mold section is shaped to form the posterior lens surface. If desired, curing of the monomeric mixture in the mold may be followed by a machining operation in order to provide a contact lens or article having a desired final configuration. Such methods are described in U.S. Pat. Nos. 3,408,429, 3,660,545, 4,113,224, 4,197,266, 5,271,875, and 5,260,000, the disclosures of which are incorporated herein by reference. Additionally, the monomer mixtures may be cast in the shape of rods or buttons, which are then lathe cut into a desired lens shape.

After recovering the contact lens from the casting operation, the contact lens is hydrated. However, in the case of silicone-containing lenses, it may be necessary to extract the lenses with an organic solvent prior to hydrating the lenses. Silicone-containing lenses generally will include contaminants, such as unreacted silicone-containing lens-forming monomers or silicone oligomers; these materials are hydrophobic and are not readily removed from the lens with aqueous solution. Accordingly, silicone-containing lenses are typically extracted with a solvent such as isopropanol, a water-miscible organic solvent, to remove these contaminants. In the subsequent hydration of silicone-containing lenses, hydration also serves to replace solvent in the lenses from the previous extraction operation with water.

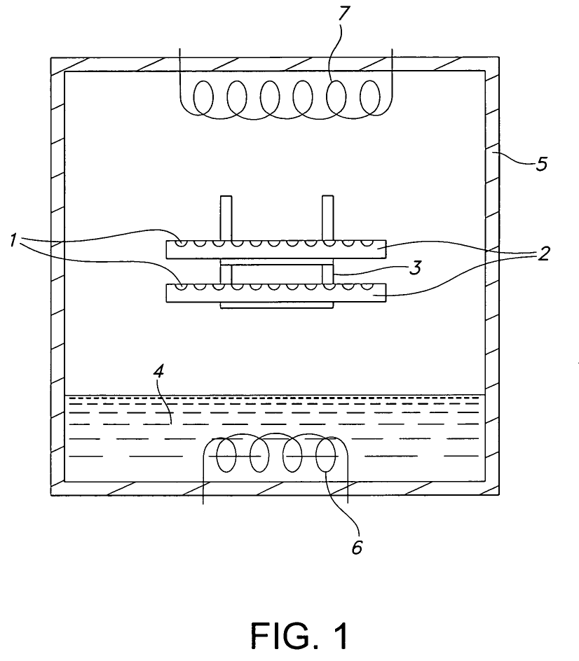

FIG. 1 illustrates schematically an apparatus and process for carrying out the invention according to various preferred embodiments.

Lenses 1 are held in trays 2, supported by holder 3. The lenses are suspended above water 4 contained in a closed vessel 5. The water 4 in vessel 5 may have the form of an aqueous solution. Water 4 is heated above its boiling point by heat source 6. Cooling coils 7 are present in the upper section of vessel 5.

Water heated by heat source 6 forms water vapor. The water vapor condenses in the upper section of vessel 5. Specifically, some vapor will condense on the lenses 1, and some vapor will condense above the lenses and drip onto lenses 1. The condensed vapors hydrate the lenses 1. Also, contaminants in or on the lenses will be washed from the lenses and returned to the lower section of vessel 5.

After the lenses are hydrated, the trays 2 may be removed from vessel 5 for additional processing. For example, in the case of contact lenses, the lenses can be packaged and sterilized. The hydrated batch of lenses may be replaced with a new batch of lenses.

Because water vapor is generated by heat source 6, it is unnecessary to use distilled water in the hydrating composition as in prior hydration processes. Since the lenses are not immersed in a hydrating composition, lenses with different chemistries may be hydrated in the same apparatus without cross-contamination; also, it is unnecessary to refresh the hydrating composition as frequently.

Various trays for holding lenses during processing are known in the art. Generally, the trays should retain the lenses so they are not misplaced during hydration in vessel; 5, and the trays should permit good circulation of water vapor about the lenses. Representative trays are described in U.S. Pat. No. 6,581,761 (Stafford et al.), and WO 03/082367 (Indra et al., US 2003/0222362 A1), the disclosures of which are incorporated herein by reference.

Having thus described the preferred embodiment of the invention, those skilled in the art will appreciate that various modifications, additions, and changes may be made thereto without departing from the spirit and scope of the invention, as set forth in the following claims.

We claim:

1. A process for hydrating biomedical devices, comprising exposing the devices to condensed water vapor.

2. The process of claim 1, comprising generating water vapor, condensing the water vapor, and exposing the devices to condensed water vapor.

3. The process of claim 2, wherein the water vapor condenses on the devices.

4. The process of claim 2, wherein vapor condenses above the devices and drips on the devices.

5. The process of claim 2, wherein the devices are suspended above heated water in a lower section of a vessel.

6. The process of claim 5, wherein a cooling mechanism is provided in an upper section of the vessel.

7. The process of claim 1, wherein said devices are ophthalmic lenses.

8. The process of claim 7, wherein said devices are contact lenses.

9. The process of claim 1, wherein the devices are formed of a hydrogel polymeric material.

10. The process of claim 9, wherein the devices are hydrogel contact lenses.

11. The process of claim 9, wherein the condensed vapor is absorbed by the polymeric material.

12. The process of claim 11, wherein excess condensed vapor washes contaminants from the device.

13. A process comprising:
heating a water source in a lower section of a vessel to generate water vapor;
suspending biomedical devices in the vessel above the water source; and
condensing the water vapor in an upper section of the vessel such that the devices are hydrated with the condensed water vapor.

14. The process of claim 13, wherein said devices are ophthalmic lenses.

15. The process of claim 14, wherein said devices are contact lenses.

16. The process of claim 13, wherein the devices are formed of a hydrogel polymeric material.

17. The process of claim 16, wherein the devices are hydrogel contact lenses.

18. The process of claim 17, wherein the condensed vapor is absorbed by the polymeric material.
